(19) United States
(12) Patent Application Publication
(10) Pub. No.: US 2009/0221967 A1
(43) Pub. Date: Sep. 3, 2009

(54) Intravascular Device

(75) Inventors: Daniel Thommen, Steinhausen (CH); Silvia Bäck-Egli, Schachen (CH); Björn Söderberg, Sävedalen (SE); Cyrill Röthlin, Hünenberg (CH); Jérôme Bernhard, Zürich (CH); Shakeel Qureshi, Surrey (GB)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: CARAG AG, Baar (CH)

(21) Appl. No.: 12/091,573

(22) PCT Filed: Oct. 28, 2004
(86) PCT No.: PCT/CH2005/000632

§ 371 Date: Mar. 3, 2009

(51) Int. Cl. A61M 5/178; A61M 31/00

(52) U.S. Cl. 604/164.09; 604/164.01; 604/500

(57) ABSTRACT

A device (1), e.g. an intravascular device, for removing an element, e.g. vascular occluding element (33), from within a lumen, e.g. a vascular lumen of a patient is described, comprising a tubular catheter body (4) made of a flexible material, with a proximal end (3), which in use is located outside of the body of the patient, and a distal end (2), which in use is located in the or at the lumen of the patient, and with a central duct (40) through which further devices (7, 14, 35) can be guided to the lumen of the patient from the outside of the patient, wherein the tubular catheter body (4) comprises a distal expansion area (25), which expansion area (25) has a contracted or folded state (26) in which the outside diameter of the expansion area (25) is substantially equal to the outside diameter of the tubular catheter body (4), and which expansion area (25) can be brought into a stiff expanded state (27) in the or at the lumen of the patient, wherein the expanded state (27) comprises an at least partially conical portion opening towards the distal end, such that an occluding element (33) can be drawn into the expansion area (25) in its expanded state (27). Such a device may be used for removal of an occluding implant which had been positioned incorrectly before or which had moved. Furthermore methods for making such a device (1) are disclosed as well as methods for using such adevice.

### TECHNICAL FIELD

The disclosure pertains to an device, e.g. to an intravascular device, for manipulations in a lumen of a patient, e.g. a vascular lumen of a patient, after insertion of the device into the patient's body. It furthermore pertains to a method for making such a device as well as to a method for using such a device for removing objects or material from a lumen of a patient.

### BACKGROUND OF THE INVENTION

In the field of intravascular surgery and medical treatment, a vast number of devices has been and is being developed for the introduction of tools into a vascular lumen of a patient, for manipulating tools in the vascular lumen of a patient, for positioning elements like occluders or stents, for removal of deposits in a lumen, for allowing image formation of specific regions of the lumen etc.

An example of such devices is the balloon catheter, as for example described in WO 2004/004821 as well as many other patent documents. Such a balloon catheter at its distal end comprises an inflatable element, the so-called balloon, which, after insertion of the catheter into the patient's body, e.g. using the femoral artery of the patient for angioplasty treatments, can be inflated by the introduction of a specific fluid, thereby stretching the artery and pressing the lesion into the artery wall thus re-establishing an acceptable blood flow through the artery.

U.S. Pat. No. 5,490,859 on the other hand for example discloses a device for the removal of intravascular occlusion material like deposits as generated in atherosclerosis. To this end, at the distal end of the proposed catheter specific structures are provided which allowed to detach such deposits and to withdraw them from the patient's body.

U.S. Pat. No. 6,488,706 describes a device for plugging an opening such as in a wall of a hollow or tubular organ, and it also describes a tool or catheter for inserting and positioning of such an occluder in the patient's body.

Usually these so-called umbrella-type occluders can only be contracted after positioning as long as it is possible to correctly attach for example a threaded portion of a tip of a catheter to a corresponding part of the occluder, and as long as it is possible to bring the occluder back into its contracted or folded state. If such a specific attachment to the occluder is not possible any more, removal or repositioning by intravascular surgery is very difficult if not impossible. In these cases therefore, invasive surgical methods have to be applied for removing or repositioning of the occluders.

### SUMMARY OF THE INVENTION

One of the objects of the present invention is therefore to provide a further device for manipulations within a lumen of a patient. Furthermore it is an object to provide a method for making such an device as well as a method for manipulating in a lumen of a patient using such a device.

The present invention proposes a device for removing an element, e.g. a vascular occluding element, from within a lumen of a patient. Such an element can e.g. be a stent, an occluder, a cage, an implant or some other device which had previously been positioned, embolized or left e.g. within the vascular lumen for example an artery or the heart of the patient. It may however also be material which has been deposited within a lumen of the patient by virtue of natural processes like for example plugs of agglutinated blood, deposits as generated in atherosclerosis, or the like. Generally it may be used in any lumen of a patient, so in a vascular lumen but also in another lumen like the stomach, a bladder or the like. Such a device comprises a tubular catheter body made of a flexible, typically plastic or metal material, with a proximal end, which in use is located outside of the body of the patient, and a distal end, which in use is located in the or at the lumen of the patient where the treatment or manipulation shall take place. Typically, on its proximal end such a tubular catheter body comprises fittings for introducing further tools or liquids and the like. The tubular catheter body therefore comprises a central duct or opening or channel through which further devices like for example manipulating catheters can be guided to the lumen of the patient from the outside of the patient. Specifically, the tubular catheter body comprises at its distal end a distal expansion area, which expansion area has a contracted or folded state in which the outside diameter of the expansion area is substantially equal to or slightly smaller than the outside diameter of the tubular catheter body, thus facilitating and allowing introduction of the device into the patient's body. This expansion area can be brought into a preferably rather stiff expanded state in the or at the lumen of the patient, so after insertion of the catheter to the lumen of the patient. The expanded state comprises an at least partially conical portion opening towards the distal end, such that e.g. an occluding element can be drawn into the expansion area in its expanded state. This specific structure allow us to introduce the folded or contracted catheter through e.g. a body vein or artery with a small diameter and using conventional systems at the introduction point of the catheter into the patient's body. The funnel-like structure at the distal end, which can be generated after insertion of the catheter to its final operating position, allows to withdraw the e.g. occluding elements into this narrowing portion, in case of a stent or an occluder usually leading to a destruction of this element but at the same time making sure that it is brought into a collapsed state and is well covered by the expansion area. Withdrawing the catheter comprising the element from the patient's body is subsequently possible easily due to the streamlined form of the expansion area.

Thus this surprisingly simple structure allows a simple and efficient removal of elements, which have to be removed for example because they have embolized, dislocated or because they have been mispositioned, because they are not necessary anymore or because they have deteriorated in some other way.

Preferably the device is an intravascular device and the lumen is a vascular lumen of a patient.

It is to be noted that the distal end of the device does not necessarily have to be located in or at the lumen where the element to be removed is located. It is also possible to introduce the device only until arriving in a vessel which is sufficiently large for the expansion area, expanding the expansion area in this vessel portion (see detailed discussion below), and introducing a removal catheter, e.g. for example the below-mentioned loop catheter, and moving with that loop catheter further into the patient's body to the lumen where the element is located. The loop catheter after having gripped the element will then withdraw the element through the vessel until arriving at the device in expanded state.

In a first preferred embodiment of the present invention, the tubular catheter body and the expansion area are one piece, typically they are a formed of the same plastic or metal tube. Is however also possible to have a first preferably plastic tube forming the tubular catheter body and to have a second element forming the expansion area. It is to be noted that the expansion area may be self-expanding, so it may be a structure, which after releasing it, automatically moves from a folded or contracted state into an unfolded or expanded state. Such a structure is for example possible by providing an expansion area which is pretensioned (elastic deformation, spring pretension, and the like). It is however preferably also possible to provide an expansion area which is expanded by an additional element, a so-called expander.

According to another preferred embodiment of the present invention, the tubular catheter body and the expansion area are tubes or, as mentioned above, preferably a single tube forming the tubular catheter body and the expansion area, made of a polymeric material. Possible materials are polyether block amides (e.g. Pebax®), polyethylene, polyamide, polytetrafluorethylene, silicone, polyvinylchloride, polyurethane, polyethyleneterephthalate, polypropylene or copolymers, combinations or mixtures thereof. To provide sufficient stiffness and at the same time sufficient flexibility, such a tube typically has a wall thickness of in the range of 0.1-0.5 mm, and for example a hardness of at least 60 Shore, preferably of at least 70 Shore. Possible is for example a tube made of Pebax® with an outer diameter of 2.5-3 mm such as to fit through standard 3.3 mm in diameter insertion coupling element (introducer), wherein the tube has a wall thickness of 0.3 mm. Typically therefore, the tubular catheter body has an outer diameter in the range of 1-4 mm, preferably of 2-3 min, and preferentially the expanded state of the expansion area has a distal opening with a diameter in the range of 4-7 mm, preferably of at least 5 mm to efficiently allow withdrawing of occluding elements into the funnel-like structure.

According to another preferred embodiment, the polymeric material of the tubular catheter body is braided, for example by polymeric strands, carbon fibre strands or metal braiding. Preferentially, in case of a one-piece catheter, only the tubular catheter body is braided but not the expansion area.

As mentioned above, preferentially the expansion area is given by a funnel-type widening portion. The axial length of the expansion area is preferably in the range of 5-50 mm, even more preferably of 15-30 mm.

Another preferred embodiment of the present invention is characterised in that the expansion area, preferably made of an unbraided polymeric tube or tube portion, is at least partly conically expanded by heat expansion or expansion by irradiation, and is folded or elastically contracted for insertion into the patient in the contracted state.

A further preferred embodiment of the (intravascular) device for insertion provides at least a dilator in the central duct, wherein said dilator comprises an at least partially conically converging dilator tip, for easy insertion of the device into the patient's body and for protecting the vessel or channel during introduction. To keep the expansion area in contracted state during introduction of the (intravascular) device, a substantially circumferential sleeve or retaining element can be provided around such a dilator tip or analogous element and/or around at least a distal portion of the contracted or folded expansion area. Preferentially said sleeve is attached to the dilator tip, such that upon pushing the dilator further into the patient's body the sleeve is removed from the distal portion of the contracted or folded expansion area thus releasing it. For example the sleeve can be a heat shrink sleeve applied prior to the insertion of the (intravascular) device it may however also be a strip of rolled around material like for example of PTFE.

As mentioned above, expansion of the expansion area may take place automatically after release for example after removal or shifting of the above-mentioned sleeve, it may however also be effected by a specific additional catheter element. So according to another preferred embodiment, further an expander catheter is provided either in addition to or as part of the dilator, said expander catheter comprising an expander which can be expanded in the lumen of the patient to bring the expansion area from the contracted or folded state into the expanded state. The expander can for example be a contraction expander expanding upon retraction of the dilator tip. A particularly simple construction is possible if as such a contraction expander a metal or plastic tube is used, which, in a distal portion, comprises axial slits, such that the stripes formed by these slits expand in a radial direction if the dilator tip is retracted with respect to the metal or plastic tube of the expander. Typically these stripes are already provided with some kind of pre-bending or kinks, such that a proper radial motion results upon retraction of the dilator tip. In the alternative, the expander can also be a balloon catheter, preferably a balloon catheter with the dilator tip provided at its distal end. In this case, expansion can be effected by filling the balloon catheter with a corresponding fluid, which, if a specific fluid like a marker fluid is used, in addition to that allow us proper positioning of the device using imaging techniques.

The present invention also relates to a (e.g. intravascular) device for removing an element from within a (e.g. vascular) lumen of a patient, comprising a tubular catheter body with a proximal end, which in use is located outside of the body of the patient, and a distal end, which in use is located in the or at the lumen of the patient, and with a central duct through which further devices can be guided to the lumen of the patient from the outside of the patient, wherein the tubular catheter body comprises a distal expansion area. This expansion area has a contracted or folded state in which the outside diameter of the expansion area is substantially equal to or smaller than the outside diameter of the tubular catheter body, and this expansion area can be brought into a substantially stiff expanded state in the or at the lumen of the patient. The expanded state comprises an at least partially conical, or funnel-like portion opening towards the distal end, such that an occluding element can be drawn or rather forced into the expansion area in its expanded state, further comprising for insertion at least a dilator in the central duct, wherein said dilator comprises an at least partially conically converging dilator tip, and a sleeve provided around said dilator tip and around at least a distal portion of the contracted or folded expansion area.

Further the present invention relates to a (e.g. intravascular) device for removing an element from within a (e.g. vascular) lumen of a patient, comprising a tubular catheter body with a proximal end, which in use is located outside of the body of the patient, and a distal end, which in use is located in the or at the lumen of the patient, and with a central duct through which further devices can be guided to the lumen of the patient from the outside of the patient, wherein the tubular catheter body comprises a distal expansion area, which expansion area has a contracted or folded state in which the outside diameter of the expansion area is substantially equal to or smaller than the outside diameter of the tubular catheter body, and which expansion area can be brought into a substantially stiff expanded state in the or at the lumen of the patient, wherein the expanded state comprises an at least partially conical portion opening towards the distal end, such that an occluding element can be drawn into the expansion area in its expanded state, further comprising for insertion at least a dilator in the central duct, wherein said dilator comprises an at least partially conically converging dilator tip, and a sleeve provided around said dilator tip and around at least a distal portion of the contracted or folded expansion area and a balloon catheter with the dilator tip at its distal end.

In addition to the above devices, the present invention also relates to a method for making such a (e.g. intravascular) device. The method is in particular characterised in that a preferably polymeric tube is provided, a distal end portion thereof is expanded by application of heat (preferably be using a heated conical pin which is pushed into the distal end portion of the tube) and/or radiation and/or softening agents under formation of a stiff expanded, at least partially conically widening state, this expanded state is folded into its folded state such as to have an outer diameter substantially equal to or smaller than the rest of the tube forming the tubular catheter body, inserting a dilator into the central duct of the tubular catheter body and the expansion area, applying a sleeve or retaining element preferably to cover parts of a dilator tip and attached thereto and/or covering or retaining preferably at least the distal portion of the expansion area in its folded state.

For the specific use of such a device, furthermore the following method for removing an element like e.g. a vascular occluding element from within a lumen, e.g. a vascular lumen, of a patient is proposed: in a first step a device, e.g. as described above, is introduced into the patient's body, wherein the device comprises a tubular catheter body with a proximal end, which in use is located outside of the body of the patient, and a distal end, which in use is located in the or at the (e.g. vascular) lumen of the patient, and with a central duct through which further devices can be guided to the lumen of the patient from the outside of the patient, wherein the tubular catheter body comprises a distal expansion area, which expansion area has a contracted or folded state in which the outside diameter of the expansion area is substantially equal to or smaller than the outside diameter of the tubular catheter body, and which expansion area can be brought into a substantially stiff expanded state in the or at the lumen of the patient, wherein the expanded state comprises an at least partially conical portion opening towards the distal end, such that an occluding element can be drawn into the expansion area in its expanded state, further comprising at least a dilator in the central duct, wherein said dilator comprises an at least partially conically converging dilator tip, and a sleeve or retaining element provided around or at said dilator tip and preferably attached thereto and/or d around or at at least a distal portion of the contracted or folded expansion area for keeping the expansion area in its contracted or folded state. In a second step, the dilator tip and the attached sleeve or retaining element is moved further into the patient's body such as to release the expansion area. In a third step, the expansion area is expanded, which may either take place automatically upon removal of the retaining element, or, as preferred, which is induced by the operator by use of an expander which is brought into its expanded state, and subsequent removal of the expander to free the distal opening of the expanded expansion area. In a fourth step, the element is gripped and drawn or forced as far as possible into the expansion area in expanded state, so into the funnel-like structure. It may even be fully or partially forced into the tubular catheter body. In the last step, the device with the enclosed element is withdrawn from the patient's body. It goes without saying that intermediate steps for further manipulations of the device or for a gripping the element, for changing the position of the element, for changing the shape of the element or the like, can be inserted at appropriate positions in the above method. Such amendments, modifications and supplementations to the above method are well within the scope of the technical knowledge of the person skilled in the art in this field. Preferably the device is an intravascular device which is used in a vascular lumen of a patient.

Further embodiments of the present invention are outlined in the dependent claims.

### SHORT DESCRIPTION OF THE FIGURES

In the accompanying drawings preferred embodiments of the invention are shown in which:

FIG. 1 a) is a side view of an intravascular device according to one aspect of the invention; b) is a detailed view of the tip portion of the intravascular device according to FIG. 1 a); is a detailed view according to FIG. 1b) with the sleeve removed;

FIG. 2 a)-d) show a possible sequence of steps using an intravascular device according to several aspects of the invention for the removal of a vascular occluding element, in this specific case an occluder;

FIG. 3 shows an intravascular device according to one aspect of the invention with expanded expansion area and removed dilator/expander;

FIG. 4 shows a dilator catheter in a side view;

FIG. 5 shows an expander catheter according to one aspect of the invention in a side view;

FIG. 6 shows an expander catheter according to another aspect of the invention in a side view; and

FIG. 7 shows a removal device in the form of a loop catheter.

### DETAILED DESCRIPTION OF THE PREFERRED EMBODIMENTS

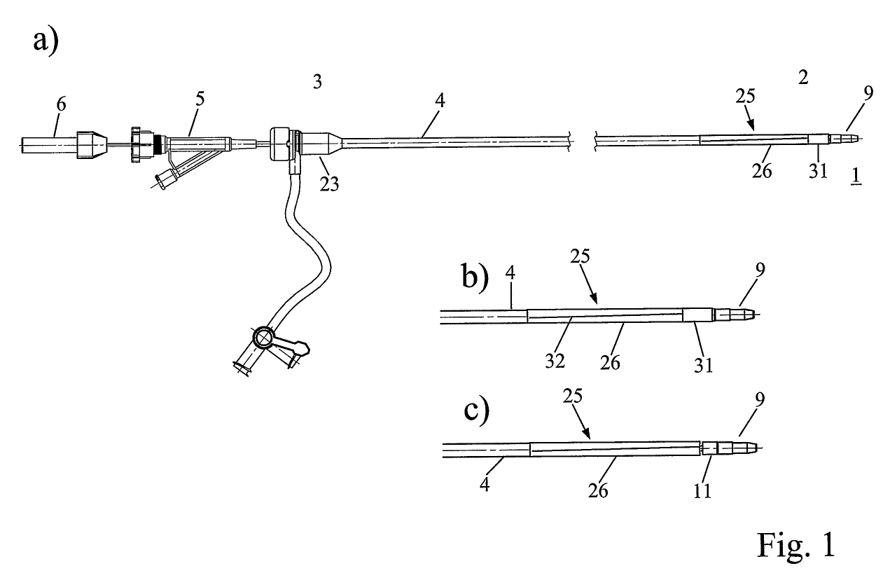

Referring to the drawings, which are for the purpose of illustrating the present preferred embodiments of the invention and not for the purpose of limiting the same, FIG. 1a shows a side view of an intravascular catheter 1. The catheter 1 is in its assembled state and in the state for insertion into the patient's body. It comprises a flexible tubular catheter body 4, which forms the main extending element of the catheter. In this specific case, the tubular catheter body 4 is given by a plastic tube made of Pebax® as e.g. available from Arkema, FR. It has an outer diameter of 2.7 mm and a wall thickness of 0.3 mm. The tubular catheter body 4 has a distal end 2 to penetrate the patient's body until the desired vascular lumen of the patient is reached, and a proximal end 3 which remains outside of the patient's body. Typically the proximal end is provided with fittings 23 which allow proper insertion of tools or fluids. At the distal end there is provided an expansion area 25, which in this case is in a folded state 26. At the very terminal tip of the distal end one can see the dilator tip 9, which is, as is the expansion area 25, partially covered by a sleeve 31 (see discussion further below).

Inserted into the central duct 40 of the catheter, there is on the one hand another tubular catheter, the expander catheter 14, and within the central duct of this expander catheter 14, there is on the other hand provided another catheter, the so-called dilator 7. At the proximal end of the expander catheter 14 there is provided with a Y-connector 5, and the dilator 7 is also provided with a fitting 6 at the proximal end for simplifying manipulation by the medical personnel.

FIG. 1b) shows a detailed view of the tip portion of the intravascular device 1. In this detailed view one can see that the expansion area 25, when in its folded state 26, comprises one or several folding lines 32, depending on how the folding has been accomplished.

One possible way of preparing the folded state is as follows: after expansion of the terminal region of the tube the expanded area is pressed onto the surface leading to two folds on both sides. Subsequently a flat stick like element is put onto this flattened portion and the two sides extending laterally from this stick are folded up. Then the stick is removed, the upfolded portions are fully bent inwards such as to have a small diameter of in the folded state.

As already pointed out above, a sleeve 31 is provided, which covers the proximal end of the dilator tip and the distal end of the expansion area 25. This circumferential sleeve provides a smooth transition between the dilator tip 9 and the expansion area 25, and it makes sure that the expansion area 25 does not expand as long as the dilator tip 9 is not moved from the position as shown in FIG. 1b). This position can more easily be recognised from the display in FIG. 1c), in which the sleeve has been removed for better visibility of the relative positioning of the tip 9 and the expansion area 25.

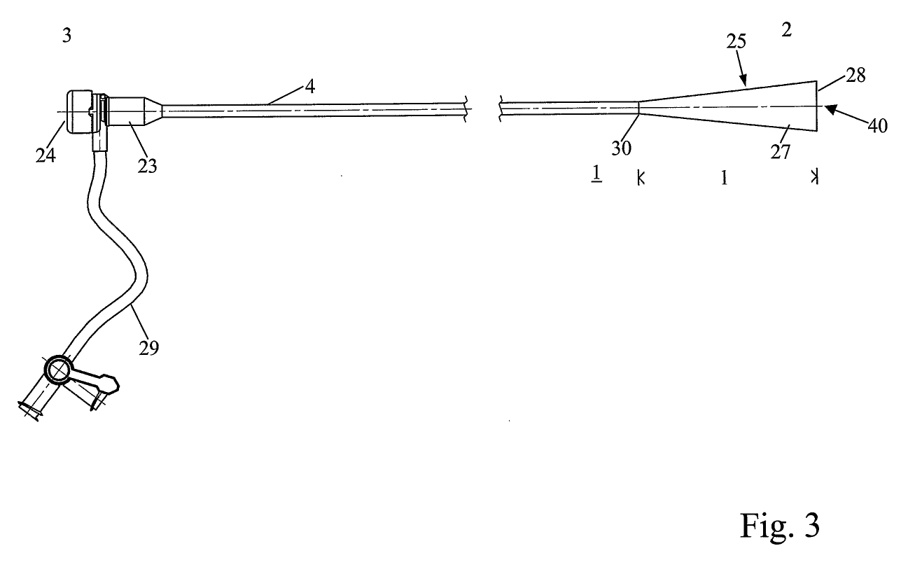

FIG. 3 shows the actual catheter 1 in a detailed view in its expanded state. The expansion area 25 is widening towards the distal end 2 of the catheter, thus forming a funnel-like structure with a large opening towards the distal end. This expansion area 25 typically has a length 1 of approximately 2-2.5 cm. It has to be pointed out that the shape of the expanded state 27 is not necessarily as displayed in FIG. 3, it may as well be a more tuba-like shape continuously and increasingly widening towards the distal end, but it may also be widening only in its proximal end portion in the region as indicated with the reference numeral 30, and it may then be almost cylindrical towards the distal end. One can see that in the expanded state there is provided a large rescue opening for any device to be withdrawn into the sheath or sleeve formed by the expansion area 25. From FIG. 3 one can also see that at the proximal end there may be provided a fitting 23 which has an opening 24 for insertion of further catheters. It may also be provided with further input possibilities 29.

The expanded state 27 of the expansion area 25 is typically formed in that a standard catheter tube made of plastic is widened for example by pushing a heated conical tip into the distal opening of the tube . If the above-mentioned material is used, a conical tip at a temperature in the range of 200-650° C. or 300-500 ° C. is sufficient for appropriate widening of this portion. For appropriate functionality of the expanded expansion area, the distal outer diameter of the expanded expansion area should be in the range of 5-50 mm, preferably 15-30 mm.

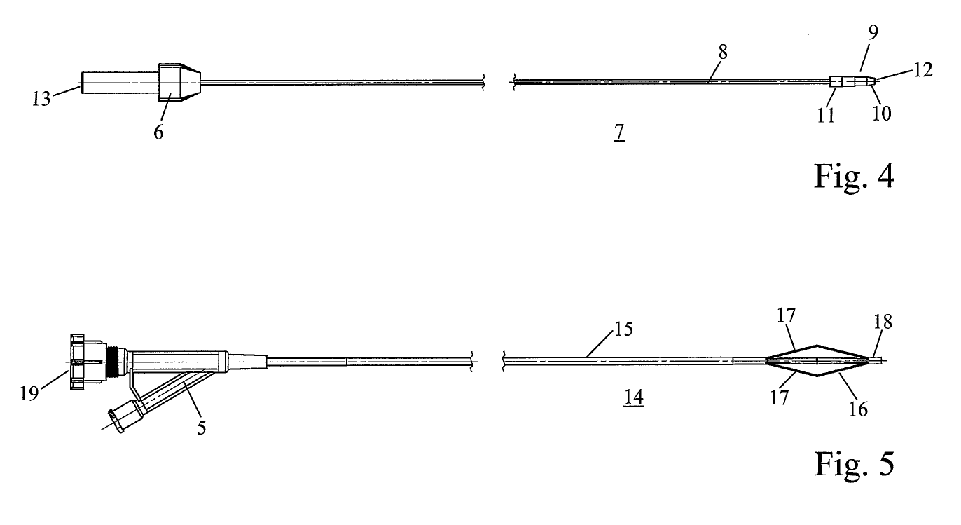

FIG. 4 shows the so called dilator 7, which basically consists of a rather stiff dilator tube 8 or dilator wire, which, at its proximal end, carries a fitting 6 which is provided with a proximal insertion opening 13 for further wires or catheters. At its distal end, the dilator 7 is provided with the above-mentioned dilator tip 9, which has a conical tip portion 10 for facilitating insertion, wherein this tip portion 10 typically is provided with a distal opening through which for example a further catheter introduced through the opening 13 can be pushed for a manipulations in the lumen of the patient. The dilator tip 9 at its proximal end comprises a body portion 11, which may be cylindrical with stepped diameters.

FIG. 5 shows one of the possible expander catheters which can be used for expanding the expansion area 25. In this case, the expander catheter 14 is given as a contraction expander which, on its proximal end carries a Y-connector 5 as a fitting, wherein this connector 5 is provided with a proximal insertion opening 19 for insertion of further catheters or wires into the expander catheter 14. The expander catheter 14 comprises an expander tube 15, which can be a metal tube or a plastic tube. In its most simple realisation, the actual contraction expander 16 is given by a distal portion of this metal or plastic tube, which is provided with axial slits such that strips 17 are formed. At the very distal end there is no slits thus forming a circumferential, ring-like distal end portion 18. If the end portion 18 is drawn towards the proximal end of the device, the strips 17 bend radially outwards. To have a clearly defined expansion, these strips 17 are typically pre-bent, in this specific case there is provided an outwardly pointing kink in the middle portion of each strip 17, making sure that maximum expansion takes place at this axial position and not staggered for each strip individually. Such an general structure expander is also new and inventive as such independent of the above intravascular device and may e.g. also be covered by a sheath to fulfil analogous functions to a balloon catheter.

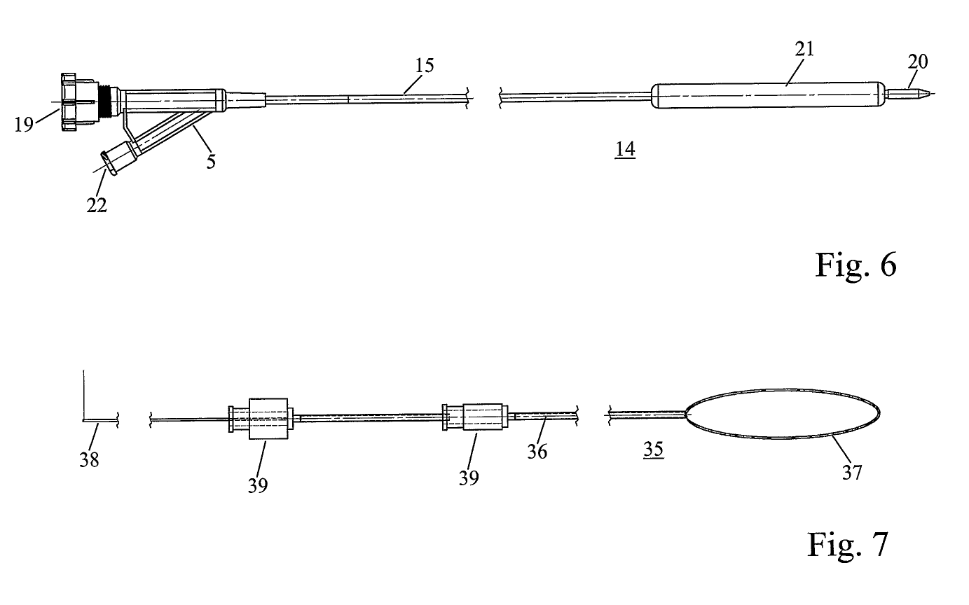

FIG. 6 shows another possibility of an expander, in this case, the expander is given as a balloon catheter. Such catheters are e.g. available from NuMed Inc. USA or from Cordis, USA. While in the above case, the dilator and the expander are two different catheter elements, in this case both functions can be taken over by the balloon catheter 14. This catheter is provided with a proximal fitting 5 as well, and in this case the internal structure of the corresponding expander tube 15 is given such that if by means of the opening 22 a fluid is inserted into the catheter, the balloon 21 provided at the distal end will inflate and expand. The balloon 21 is thus provided as a multilayered structure. At the very distal end of the catheter, substantially adjacent to the balloon 21, there is provided a dilator tip 20 which in this case takes over the function of the above-mentioned dilator tip 9.

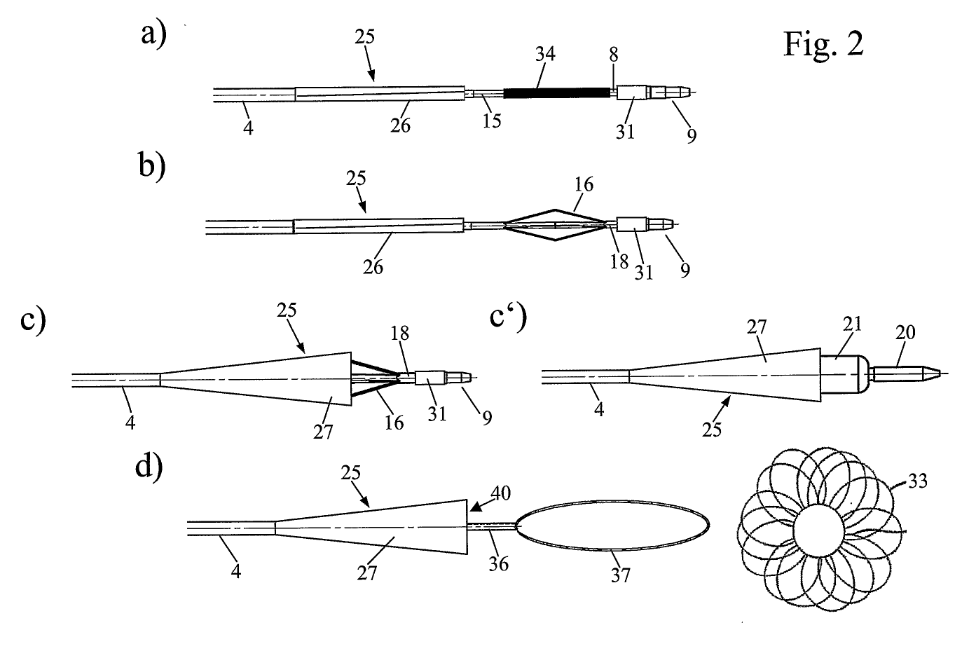

One of the possible uses of the above-mentioned intravascular devices is illustrated stepwise in FIG. 2. An intravascular device as displayed in FIG. 1 a) is first introduced into the patient's body until the distal end 2 arrives at the desired vascular lumen of the patient. In this moment, as is indicated in FIG. 2a), the two fittings 5 and 6 are pushed towards the fitting 23, such that that the expander 14 as well as the dilator 7 are pushed out of the flexible tubular catheter body 4 and out of the expansion area 25. Since the sleeve 31 is attached to the dilator tip, it will be shifted off the distal end portion of the expansion area 25 thus releasing this folded area. Now the two fittings 5 and 6 are pushed towards each other, wherein the contraction expander 16 expands as indicated in FIG. 2b). Now both fittings 5 and 6 are synchronously pushed back and away from the fitting 23 such that the expander 16 is pushed into the expansion area 25, leading to a situation as displayed in FIG. 2c). It is also possible to expand the expander 16 while it is located within the expansion area 25.

The above method is shown for the use of a contraction expander. For the sake of completeness, FIG. 2c′) shows the situation analogous to the display in FIG. 2c) if a balloon catheter 21 is used.

After having expanded the expansion area 25 into the expanded state 27, the expander 14 is completely removed out of the patient's body, and instead another catheter is inserted for gripping for example an occluder or implant 33. As indicated in FIG. 2d) this can for example be a loop catheter 35.

Such a loop catheter 35 is detailed in FIG. 7. It has a loop catheter tube 36 which, on its proximal end is provided with fittings 39. Within this loop catheter tube 36 there is provided a wire, which, if pushed out of the distal end of the loop catheter tube 36 forms the actual loop 37. This wire can be activated by means of a handle 38 which protrudes out of the fittings 39. Upon manipulation of the handle 30 it is possible to change the position of the loop 37, to change its size etc.

Returning now to FIG. 2 d), such a loop catheter 35 is introduced into the flexible tubular catheter body until it protrudes out of the distal end of the expanded portion 27. Subsequently, the actual loop 37 is brought into a proper position to catch the e.g. embolized implant 33, the size of the loop is reduced until the implant 33 is caught, and subsequently the implant 33 is forced into the expanded expansion area 27. In this moment typically the implant 33 destructively and irreversibly collapses and is forced into the sleeve 27. After the implant is buried within this sleeve 27, it is then safe to retract the intravascular device encapsulating the collapsed implant 33 from the patient's body.

### LIST OF REFERENCE NUMERALS

1 catheter

2 distal end of catheter

3 proximal end of catheter

4 flexible tubular catheter body

5 Y-connector of expander

6 fitting of dilator

7 dilator

8 dilator tube

9 dilator tip

10 conical tip portion of 9

11 body portion of 9

12 distal opening of 9

13 proximal insertion opening of 6

14 expander catheter

15 expander catheter tube

16 contraction expander

17 wires/strips of 16

18 distal end portion of 14

19 proximal insertion opening of 14

20 dilator tip on balloon expander

21 balloon expander

22 input opening for fluid for expanding 21

23 fitting of catheter body

24 proximal insertion opening of 1

25 expansion area of 4

26 25 in contracted or folded state

27 25 in expanded state

28 rescue opening

29 additional input on 23

30 proximal end of 27

31 sleeve

32 folding line of 25

33 implant

34 contraction expander, contracted state

35 loop catheter

36 loop catheter tube

37 loop

38 handle of 35

39 fittings of 35

40 central duct in 4

1. Device (1) for removing an element (33) like a vascular occluding element from within a lumen of a patient, comprising a tubular catheter body (4) made of a rigid or flexible material, with a proximal end (3), which in use is located outside of the body of the patient, and a distal end (2), which in use is located in the or at the lumen of the patient, and with a central duct (40) through which further devices (7, 14, 35) can be guided to the lumen of the patient from the outside of the patient, wherein the tubular catheter body (4) comprises a distal expansion area (25), which expansion area (25) has a contracted or folded state (26) in which the outside diameter of the expansion area (25) is substantially equal to the outside diameter of the tubular catheter body (4), and which expansion area (25) can be brought into a stiff expanded state (27) in the or at the lumen of the patient, wherein the expanded state (27) comprises an at least partially conical portion opening towards the distal end, such that an occluding element (33) can be drawn into the expansion area (25) in its expanded state (27).

2. Device (1) according to claim 1, wherein the tubular catheter body (4) and the expansion area (25) are one piece.

3. Device (1) according to claims 1 or 2, wherein the tubular catheter body (4) and the expansion area (25) are tubes or a single tube made of a polymeric material, preferably of polyether block amides, polyethylene, polyamide, polytetrafluorethylene, silicone, polyvinylchloride, polyurethane, polyethyleneterephthalate, polypropylene, or copolymers, combinations or mixtures thereof.

4. Device (1) according to claim 3, wherein the polymeric material of the tubular catheter body (4) is braided, preferably by polymeric, carbon fibre or metal braiding.

5. Device (1) according to claim 1, wherein the tubular catheter body (4) has an outer diameter in the range of 1-4 mm, preferably of 2-3 mm, and wherein the expanded state (27) of the expansion area (25) has a distal opening with a diameter in the range of 4-7 mm, preferably of at least 5 mm.

6. Device (1) according to claim 1, wherein the expansion area (25) is given by a funnel-type widening portion with an axial length (1) in the range of 5-50 mm, preferably of 15-30 mm.

7. Device (1) according to claim 1, wherein the expansion area (25) is made of a preferably unbraided polymeric tube, conically expanded by heat expansion, and folded for insertion into the patient.

8. Device (1) according to claim 1, wherein for insertion at least a dilator (7, 14) is provided in the central duct (40), wherein said dilator (7, 14) comprises an at least partially conically converging dilator tip (9, 20).

9. Device (1) according to claim 8, wherein a substantially circumferential sleeve (31) is provided around said dilator tip (9, 20) and around at least a distal portion of the contracted or folded expansion area (26), wherein preferentially said sleeve (31) is attached to the dilator tip (9, 20).

10-15. (canceled)

16. Device (1) according to claim 8, wherein a substantially circumferential sleeve (31) is provided around said dilator tip (9, 20) and around at least a distal portion of the contracted or folded expansion area (26), wherein preferentially the sleeve (31) is a heat shrink sleeve applied prior to the insertion of the device.

17. Device (1) according to claim 8, wherein further an expander catheter (14) is provided either in addition to or as part of the dilator (7, 14), said expander catheter (14) comprising an expander (16, 21) which can be expanded in the lumen of the patient to bring the expansion area (25) from the contracted or folded state (26) into the expanded state (27).

18. Device (1) according to claim 17, wherein the expander (16) is a contraction expander expanding upon retraction of the dilator tip (9) or wherein the expander (21) is a balloon catheter, preferably a balloon catheter (21) with the dilator tip (20) at its distal end.

19. Device (1) for removing an element (33) from within a lumen of a patient, comprising a tubular catheter body (4) with a proximal end (3), which in use is located outside of the body of the patient, and a distal end (2), which in use is located in the or at the lumen of the patient, and with a central duct (40) through which further devices (7, 14, 35) can be guided to the lumen of the patient from the outside of the patient, wherein the tubular catheter body (4) comprises a distal expansion area (25), which expansion area (25) has a contracted or folded state (26) in which the outside diameter of the expansion area (25) is substantially equal to or smaller than the outside diameter of the tubular catheter body (4), and which expansion area (25) can be brought into a substantially stiff expanded state (27) in the or at the lumen of the patient, wherein the expanded state (27) comprises an at least partially conical portion opening towards the distal end, such that an occluding element (33) can be drawn into the expansion area (25) in its expanded state (27), further comprising for insertion at least a dilator (7, 14) in the central duct (40), wherein said dilator (7, 14) comprises an at least partially conically converging dilator tip (9, 20), and a sleeve (31) provided around said dilator tip (9, 20) and around at least a distal portion of the contracted or folded expansion area (26).

20. Device (1) for removing an element (33) from within a lumen of a patient, comprising a tubular catheter body (4) with a proximal end (3), which in use is located outside of the body of the patient, and a distal end (2), which in use is located in the or at the lumen of the patient, and with a central duct (40) through which further devices (7, 14, 35) can be guided to the lumen of the patient from the outside of the patient, wherein the tubular catheter body (4) comprises a distal expansion area (25), which expansion area (25) has a contracted or folded state (26) in which the outside diameter of the expansion area (25) is substantially equal to or smaller than the outside diameter of the tubular catheter body (4), and which expansion area (25) can be brought into a substantially stiff expanded state (27) in the or at the lumen of the patient, wherein the expanded state (27) comprises an at least partially conical portion opening towards the distal end, such that an occluding element (33) can be drawn into the expansion area (25) in its expanded state (27), further comprising for insertion at least a dilator (7, 14) in the central duct (40), wherein said dilator (7, 14) comprises an at least partially conically converging dilator tip (9, 20), and a sleeve (31) provided around said dilator tip (9, 20) and around at least a distal portion of the contracted or folded expansion area (26) and a balloon catheter (21) with the dilator tip (9, 20) at its distal end.

21. Method for making a device (1), wherein a polymeric tube is provided, a distal end portion thereof is expanded by application of heat and/or radiation and/or softening agents under formation of a stiff expanded state (27), this expanded state is folded into its folded state (26) such as to have an outer diameter substantially equal to or smaller than the rest of the tube forming the tubular catheter body (4), inserting a dilator (7, 14) into the central duct of the tubular catheter body (4) and the expansion area (25), applying a sleeve (31) to cover parts of a dilator tip (9, 20) and at least the distal portion of the expansion area (25) in its folded state (26).

22. Method for removing an element (33) like a vascular occluding element from within a lumen like a vascular lumen of a patient, comprising the steps of introducing a device (1) into the patient's body, wherein the device comprises a tubular catheter body (4) with a proximal end (3), which in use is located outside of the body of the patient, and a distal end (2), which in use is located in the or at the lumen of the patient, and with a central duct (40) through which further devices (7, 14, 35) can be guided to the lumen of the patient from the outside of the patient, wherein the tubular catheter body (4) comprises a distal expansion area (25), which expansion area (25) has a contracted or folded state (26) in which the outside diameter of the expansion area (25) is substantially equal to or smaller than the outside diameter of the tubular catheter body (4), and which expansion area (25) can be brought into a substantially stiff expanded state (27) in the or at the lumen of the patient, wherein the expanded state (27) comprises an at least partially conical portion opening towards the distal end, such that an occluding element (33) can be drawn into the expansion area (25) in its expanded state (27), further comprising at least a dilator (7, 14) in the central duct (40), wherein said dilator (7, 14) comprises an at least partially conically converging dilator tip (9, 20), and a sleeve (31) provided around said dilator tip (9, 20) and around at least a distal portion of the contracted or folded expansion area (26),
of moving the dilator tip (9, 20) and the attached sleeve (31) further into the patient's body such as to release the expansion area (25)
expanding the expansion area (25), preferably by use of an expander (16, 21) into its expanded state (27), and subsequent removal of the expander (16, 21)
removing the element (33) by gripping it and by drawing it as far as possible into the expansion area (25) in expanded state (27) and possibly into the tubular catheter body (4), withdrawing the device (1) with the enclosed element (33) from the patient's body.
